(19) United States
(12) Patent Application Publication
(10) Pub. No.: US 2025/0302766 A1
(43) Pub. Date: Oct. 2, 2025

(54) FORMULATIONS FOR ORAL DELIVERY OF POLYPEPTIDES, ANTIBODIES AND PROTEINS AND USES THEREOF

(71) Applicant: PRODIGY BIOTECH, INC., West Chester, PA (US)

(72) Inventors: Vivek Gupta, Floral Park, NY (US); Vineela Parvathaneni, Greenville, NC (US); Sruthi Sarvepalli, Newark, NJ (US)

(73) Assignee: PRODIGY BIOTECH, INC., West Chester, PA (US)

(21) Appl. No.: 18/864,232

(22) PCT Filed: May 11, 2023
(86) PCT No.: PCT/US2023/021842

§ 371 Date: Nov. 8, 2024

Related U.S. Application Data
(60) Provisional application No. 63/341,727, filed on May 13, 2022.

(51) Int. Cl. A61K 9/51; A61K 9/00; C07K 16/02

(52) U.S. Cl. CPC A61K 9/5138; A61K 9/0053; A61K 9/5161; A61K 9/5192; C07K 16/02

(57) ABSTRACT

Provided is a nanoparticle comprising a composition comprising a polypeptide having a molecular weight greater than 50,000 g/mol (e.g. IgY antibodies), wherein the composition is encapsulated in a material that includes a biocompatible bioerodible polymer. Also provided is a method of preparing these nanoparticles, and use of the nanoparticles as a therapeutic to treat a disease condition.

### RELATED APPLICATIONS

This application claims priority to U.S. Provisional Patent Application No. 63/341,727 filed on May 13, 2022, the contents of which are incorporated by reference herein in their entirety.

### TECHNICAL FIELD

The present invention relates to nanoparticles comprising a composition that includes a polypeptide having a molecular weight greater than 50,000 g/mol (e.g. antibodies, such as IgY antibodies, and proteins), wherein the composition is encapsulated in a material that includes a biocompatible bioerodible polymer. Also provided is a method of preparing these nanoparticles, and use of the nanoparticles as a therapeutic to treat a disease condition.

### BACKGROUND

One of the challenges for oral delivery of therapeutic proteins such as IgY antibodies is that most proteins have shorter life spans and negligible absorption in the gastrointestinal tract. Accordingly, a need exists for improved formulations of therapeutic proteins such as IgY antibodies for oral delivery.

### SUMMARY OF THE INVENTION

In certain aspects, the disclosure relates to a nanoparticle comprising a composition comprising a polypeptide, an antibody, or a protein, wherein the composition is encapsulated in a material containing a biocompatible bioerodible polymer and a surface-active polymer. In certain embodiments, the biocompatible bioerodible polymer is a polymethacrylate. In certain embodiments, the nanoparticle has a diameter that is less than 300 nm. In certain embodiments, the nanoparticle comprises at least 5% w/w of the polypeptide. In certain embodiments, the polypeptide is IgY. In certain embodiments, the IgY is obtained from a hyperimmunized egg. In certain embodiments, the composition is a hyperimmunized egg product. In certain embodiments, the composition is a defatted fraction from egg yolk comprising at least 95% IgY by weight. In certain embodiments, the composition is purified IgY.

In certain aspects, the disclosure relates to a pharmaceutical composition comprising a nanoparticle as described herein and a pharmaceutically acceptable carrier.

In certain embodiments, the pharmaceutically acceptable carrier is suitable for oral administration. The pharmaceutical composition can be in a form selected from the group consisting of a powder, a tablet, an enterically coated tablet, a capsule, an enterically coated capsule, a suspension, a solution, and an oral beverage. A substance that forms a controlled-release coating can be used for providing as enteric coating to yield an enterically coated tablet or capsule. For example, the substance can be one or more selected from the group consisting of hydroxypropyl methylcellulose phthalate, hydroxymethylethyl cellulose phthalate, hydroxypropyl methylcellulose acetate succinate, carboxymethylethyl cellulose, a methacrylic acid-methyl methacrylate copolymer (e.g., EUDRAGIT® L 100 and EUDRAGIT® Si 00, Evonik), and a methacrylic acid-ethyl acrylate copolymer (e.g., EUDRAGIT® L 100-55 and EUDRAGIT® L 30D55, Evonik).

In certain aspects, the disclosure relates to a method of delivering a polypeptide to a subject, comprising administering the nanoparticles containing a polypeptide or a pharmaceutical composition comprising nanoparticles containing a polypeptide as described herein to the subject by oral administration. In certain embodiments of the aforementioned methods, the polypeptide is IgY. In certain embodiments, the IgY is obtained from a hyperimmunized egg. In certain embodiments, the composition in the nanoparticles is a hyperimmunized egg product. In certain embodiments, the composition in the nanoparticles is a defatted fraction from egg yolk comprising at least 95% IgY by weight. In certain embodiments, the composition contained in the nanoparticles includes a purified IgY. In certain embodiments, the biocompatible bioerodible polymer of the nanoparticle includes a polymethacrylate. In certain embodiments, the nanoparticle has a diameter that is less than 300 nm. The particles can be used for oral delivery of a polypeptide. The nanoparticles can be included in a tablet, a capsule, a suspension, an emulsion, or a beverage for oral administration. In certain embodiments, the nanoparticle comprises at least 5 wt % of the polypeptide based on the total weight of the nanoparticles.

In certain embodiments, the nanoparticle further comprises a surface-active polymer. The surface-active polymer can be selected from among poly(vinyl) alcohol (PVA), polyvinylpyrrolidone (PVP), and combinations thereof.

Also provided is a method of medical treatment comprising administering to a patient an effective amount of the nanoparticles containing a polypeptide.

Also provided is a method of producing nanoparticles comprising a polypeptide having a molecular weight greater than 50,000 g/mol, the method including mixing a solvent with a polypeptide to form a mixture, mixing the mixture with a biocompatible bioerodible polymer, a surface-active polymer, and an organic solvent, carrying out an emulsification to produce an emulsion, high pressure homogenizing the emulsion at a pressure of at least 10,000 psi, and evaporating the solvents to leave the nanoparticles, wherein surface-active polymer is employed to stabilize the emulsion.

Also provided are nanoparticles that contain a composition comprising a polypeptide, where the composition is encapsulated in a material comprising a biocompatible bioerodible polymer and a surface-active polymer. The biocompatible bioerodible polymer can include a polymethacrylate. The biocompatible bioerodible polymer can include methacrylic acid-methyl methacrylate copolymer (1:1) or methacrylic acid-methyl methacrylate copolymer (1:2), or a combination thereof. The nanoparticle can have a diameter that is less than 300 nm. The nanoparticle can include or contain at least 5 wt % of the polypeptide based on the total weight of the nanoparticle. The polypeptide of the nanoparticle can be IgY. The IgY can be obtained from a hyperimmunized egg. The composition within the nanoparticle can be a hyperimmunized egg product. The composition within the nanoparticle can be a defatted fraction from egg yolk comprising at least 95% IgY by weight. The composition within the nanoparticle can be or include a purified IgY. In the nanoparticles provided herein, the surface-active polymer can be selected from among poly(vinyl) alcohol (PVA), polyvinylpyrrolidone (PVP), and combinations thereof.

Also provided are methods of delivering a polypeptide, antibody, or protein to a subject, the method including administering a pharmaceutical composition that include a nanoparticle as described herein to the subject by oral administration. The polypeptide contained in the nanoparticle can be IgY. The IgY can be obtained from a hyperimmunized egg. The nanoparticles can contain a composition that includes a hyperimmunized egg product. The nanoparticles can contain a composition that includes a defatted fraction from egg yolk comprising at least 95% IgY by weight. The nanoparticles can contain a composition that includes a purified IgY. In the methods, the delivered nanoparticle can include a biocompatible bioerodible polymer. The biocompatible bioerodible polymer can include a polymethacrylate. The biocompatible bioerodible polymer can include methacrylic acid-methyl methacrylate copolymer (1:1) or methacrylic acid-methyl methacrylate copolymer (1:2), or a combination thereof. The nanoparticles can have a diameter that is less than 300 nm. The nanoparticle can include at least 5% w/w of the polypeptide based on the total weight of the nanoparticle.

Also provided are methods of preparing the nanoparticle as described herein. The method includes (a) preparing a first mixture comprising a composition comprising a polypeptide, water, and a surface-active polymer; (b) sonicating the first mixture; (c) adding a biocompatible bioerodible polymer to ethanol and sonicating to produce a second mixture; (d) mixing the first mixture and second mixture with an organic solvent to form a third mixture; (e) homogenizing the third mixture using a probe homogenizer at a speed of about 5,000 to 15,000 rpm to yield a coarse emulsion; (f) subjecting the coarse emulsion to high pressure homogenization using a high-pressure homogenizer at a pressure of about 10,000 psi to 30,000 psi to form a fourth mixture; and (g) evaporating the ethanol and the organic solvent from the fourth mixture to yield a suspension containing the nanoparticle. The method also can include as a step centrifuging the suspension to obtain the nanoparticle. In the methods, the surface-active polymer can be selected from among poly(vinyl) alcohol (PVA), polyvinylpyrrolidone (PVP), and combinations thereof. The organic solvent can include absolute ethanol, dichloromethane (DCM), acetonitrile, acetone, ethyl acetate, chloroform, and combinations thereof. In some methods, the organic solvent includes dichloromethane. The first mixture can include from about 2 wt % to 5 wt % of the polypeptide, and the polypeptide can be IgY. The second mixture can include from 10 mg/mL to 20 mg/mL of the biocompatible bioerodible polymer. In some embodiments, the surface-active polymer comprises 0.5 to 2.5 wt % poly(vinyl) alcohol (PVA), or 0.5 to 2.5 wt % polyvinylpyrrolidone (PVP), or a combination thereof. The first mixture can include from 0.75 wt % to 2 wt % PVA.

### BRIEF DESCRIPTION OF THE DRAWINGS

FIG. 1 is a graph illustrating a calibration curve for IgY quantification.

### DETAILED DESCRIPTION AND PREFERRED EMBODIMENTS

In certain aspects, the present disclosure relates to a nanoparticle that contains a composition comprising a polypeptide, where the composition is encapsulated in a biocompatible bioerodible polymer and a surface-active polymer exhibiting at least weak surface activity. Examples of a surface-active polymer include polyvinyl alcohol (PVA), polyvinylpyrrolidone (PVP), and combinations thereof. Applicants have shown that a polypeptide having a molecular weight greater than 50,000 g/mol, or greater than 100,000 g/mol, such as IgY, encapsulated in a nanoparticle comprising a biocompatible bioerodible polymer (such as EUDRAGIT® L 100 or EUDRAGIT® S 100, (Evonik Industries AG, Essen, Germany)) or a combination thereof, and a surface-active polymer remains intact through the encapsulation process, and have identified encapsulation methods that result in reduced nanoparticle size and high drug loading of the nanoparticles. In a particular embodiment, the composition that is encapsulated is a hyperimmunized egg product, and the polypeptide is IgY antibody. In some embodiments, the nanoparticles contain a composition that includes a purified IgY antibody.

### Definitions

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a,” “an” and “the” include plural referents unless the context clearly dictates otherwise.

As used herein, all ranges include the upper and lower limits. As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.3-4.4, etc.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics. All of the values characterized by the above-described modifier “about,” are also intended to include the exact numerical values associated therewith. Hence “about 5 percent” means “about 5 percent” and also “5 percent.”

As used herein, the terms “comprises” and “comprising” are inclusive and open ended, and not exclusive. When used in the specification and claims, the terms “comprises” and “comprising” and variations thereof mean the specified features, steps or components are included, but do not exclude other features, steps or components.

Any compositions described herein are intended to encompass compositions which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, the term “exemplary” means “serving as an example or illustration,” and should not be construed as being preferred or advantageous over other configurations disclosed herein.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

As used herein, the term “subject” includes members of the animal kingdom including but not limited to human beings.

As used herein, “weight percent” or “wt %” refers to the concentration of a substance as the weight of that substance divided by the total weight of the composition and multiplied by 100.

The term “hyperimmunization” means repeated exposure to one or more antigens such that an immune response is elevated and maintained above the natural unexposed state.

A “hyperimmune state” refers to an elevated immune response in an egg producing animal that has been hyperimmunized.

The term “egg” as used herein refers to a whole egg (table, hyperimmunized or otherwise). The term “egg product” as used herein refers to a whole egg or any product or fraction obtained from a whole egg. In a particular embodiment, the egg product is an egg yolk, for example, an egg yolk powder. In another embodiment, the egg product is an egg white, for example, an egg white powder. In another embodiment, the egg product is obtained from a whole egg, for example, a whole egg powder (e.g. a spray-dried whole egg powder).

The term “control egg” refers to an egg obtained from an egg-producing that is not maintained in a hyperimmunized state, i.e. an animal that has not been hyperimmunized. The term “control egg product” refers to a control egg or an egg product obtained from a control egg.

The term “hyperimmunized egg” refers to a whole egg obtained from an egg-producing animal maintained in a hyperimmune state, i.e. an egg-producing animal that has been hyperimmunized. The term “hyperimmunized egg product” refers to a hyperimmunized egg or any product obtained from a hyperimmunized egg.

In certain embodiments, the hyperimmunized egg product is a concentrate. As used herein the term “concentrate” refers to a hyperimmunized egg product that is at least partially purified, such that the concentration of antibodies in the concentrate is greater than the concentration of antibodies in a hyperimmunized egg.

The term “egg powder” refers to a whole egg that has been dried. In some embodiments, the egg powder is spray-dried.

The term “egg-producing animal” means any oviparous animal, and includes any animal that lays an egg, such as avians, fish and reptiles.

The term “avian” refers to an animal that is a member of the class Aves. Avians include, but are not limited to, chickens, turkeys, geese, ducks, pheasants, quail, pigeons and ostriches.

The term “administer” means any method of providing a subject with a substance, including orally, intranasally, parenterally (intravenously, intramuscularly, or subcutaneously), rectally, topically or intraocularly.

The term “antigen” refers to a substance that is able to induce a humoral antibody and/or cell-mediated immune response rather than immunological tolerance. The term signifies the ability to stimulate an immune response as well as react with the products of it, e.g., an antibody.

As used herein, an “antibody” is a protein that includes at least one complementarity determining region that binds to a specific target antigen. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. In a particular embodiment, the antibody is a polyclonal antibody. In a particular embodiment, the antibody is an IgY antibody.

As used herein, “IgY” refers to one or more than one IgY antibody.

The term “polyclonal antibody”, as used herein, refers to a population of antibody molecules that that are capable of immunoreacting with different epitopes on a particular antigen.

As used herein, “nanoparticle” refers to a particle or a structure in the nanometer (nm) range, typically from about 1 to about 1000 nm in diameter.

As used herein, the term “percent loading” refers to a ratio of the weight of polypeptide, such as IgY antibody, to the weight of a nanoparticle, multiplied by 100.

As used herein, “dalton” is a unit of molecular weight abbreviated as Da, and 1 Da equals 1 g/mol.

As used herein, a “polypeptide” refers to a polymer that includes a plurality of amino acid residue bonded together, and having a molecular weight of at least 50,000 Da (g/mol) or more.

As used herein, a “protein” refers to a polymer that includes a plurality of amino acid residue bonded together, and having a molecular weight of at least 50,000 Da (g/mol) or more.

The IgY antibody molecule has a structure similar to that of IgG antibody, with two heavy chains (H), each one with a molecular weight of about 67 to 70 kDa, and two light chains (L), with a molecular weight of about 25 kDa

As used herein, a “probe homogenizer” refers to a rotor/stator type homogenizer, that includes a rotating blade member (rotor) and a stationary collar that contains perforations (stator).

As used herein, a “high-pressure homogenizer” refers to homogenizing device that include a homogenization valve that typically includes a narrow channel through which a material is forced to flow, and a high-pressure pump that forces the material through the valve. A high-pressure homogenizer typically operates at a pressure from 5,000 psi to 45,000 psi.

### Hyperimmunized Egg Product

Egg-producing animals produce antibodies in blood and eggs that are specific to particular immunogens. For example, various genera of the class Aves, such as chickens (Gallus domesticus), turkeys, and ducks produce antibodies against antigens associated with avian diseases. LeBacq-Verheyden et al. (Immunology 27:683 (1974)) and Leslie, G. A., et al. (J. Med. 130:1337 (1969)), have quantitatively analyzed immunoglobulins of the chicken. Polson, A., et al. (Immunological Communications 9:495-514 (1980)) immunized hens against several proteins and natural mixtures of proteins, and detected IgY antibodies in the yolks of the eggs. Fertel, R., et al. (Biochemical and Biophysical Research Communications 102:1028-1033 (1981)) immunized hens against prostaglandins and detected antibodies in the egg yolk. Jensenius et al. (Journal of Immunological Methods 46:63-68 (1981)) provide a method of isolating egg yolk IgG for use in immunodiagnostics. Polson et al. (Immunological Communications 9:475-493 (1980)) describe antibodies isolated from the yolk of hens that were immunized with a variety of plant viruses.

U.S. Pat. No. 4,748,018 (Stolle et al., 1988) discloses a method of passive immunization of a mammal that comprises parenterally administering purified antibody obtained from the eggs of an avian that has been immunized against the corresponding antigen, and wherein the mammal has acquired immunity to the eggs. U.S. Pat. No. 5,772,999, (Greenblatt et al., 1998), discloses a method of preventing, countering or reducing chronic gastrointestinal disorders or Non-Steroidal Anti-Inflammatory Drug-induced (NSAID-induced) gastrointestinal damage in a subject by administering hyperimmunized egg and/or milk or fractions thereof to the subject.

An immunized egg is an egg which comes from an avian which has been immunized with, for example, a specific antigen or mixture of antigens. A hyperimmunized egg is an egg which comes from an avian which has been brought to a specific state of immunization by means of, for example, periodic booster administrations of antigens. Hyperimmunized eggs, no matter the type of antigen their avian maker has been administered, have been found to have various beneficial factors, including, as mentioned above, the treatment of chronic gastrointestinal disorders, NSAID-induced gastrointestinal damage (see U.S. Pat. No. 5,772,999) and anti-inflammatory effects due to the presence of an anti-inflammatory composition (see U.S. Application Publication No. US 2004/0156857 (Adalsteinsson et al., 2004)).

One of the advantages of the hyperimmunized egg product is that it would have a higher and more consistent level of antibodies (e.g. IgY antibodies) to one or more of the antigens described herein compared to a control egg product or an egg product from a chicken that has been immunized with the antigen using standard immunization techniques. Typically standard immunization consists of an initial immunization followed by one or two booster immunization at 30 day intervals. In some embodiments, hyperimmunization comprises at least 4, 5, 6, 7, 8, 9 or 10 immunizations with an antigen described herein. In some embodiments, hyperimmunization comprises immunizing an egg producing animal with an antigen described herein at intervals of less than 30 days, less than 25 days, less than 20 days, less than 15 days, less than 10 days, or less than 5 days. In some embodiments, hyperimmunization comprises immunizing an egg producing animal with an antigen described herein at an interval of 1 week, 2 weeks, 3 weeks, 4 weeks, 1 month, 2 months or 3 months. Any of these values can be used to define a range for the interval at which the egg producing animal is immunized. For example, in some embodiments, the egg producing animal is hyperimmunized at an interval ranging from once every 2 weeks to once every 3 months, once per week to once every 3 months, or once every 2 weeks to once per month.

The hyperimmunized egg product can be produced by any egg-producing animal. It is preferred that the animal be a member of the class Aves or, in other words, an avian. Within the class Aves, domesticated fowl are preferred, but other members of this class, such as turkeys, ducks, and geese, are a suitable source of hyperimmune egg product. In a particular embodiment, the egg-producing animal is a chicken.

This special state of hyperimmunization is preferably achieved by administering an initial immunization, followed by periodic boosters with sufficiently high doses of specific antigens or mixtures of antigens. The dosage of the booster can be 10%, 20%, 30%, 40%, 50%, 60%, 70%, 80%, 90% or 100% of the dosage necessary to produce primary immunization of the egg-producing animal. Any of these percentages can be used to define a range for the dosage of the booster immunization. For example, in some embodiments, the dosage of the booster is 20%-80%, 30%-70%, or 50%-100% of the dosage necessary to produce primary immunization of the egg-producing animal. In a particular embodiment, the dosage of the booster immunization is 50% of the dosage of the primary immunization.

Having knowledge of the requirement for developing and maintaining a hyperimmune state, it is within the skill of the art to vary the amount of antigen administered, depending on the egg-producing animal genera and strain employed, in order to maintain the animal in the hyperimmune state.

The hyperimmune state can be produced by a single antigen or a combination of antigens. Hyperimmunization can be achieved by multiple exposures to multiple antigens, or multiple exposures to a single antigen.

### Hyperimmunization Procedure

The following list of steps is an example of a preferred procedure used to bring an egg-producing animal to a heightened state of immunity from which the resultant hyperimmune egg or egg product can be administered to an avian:
1. Selecting one or more antigens.
2. Eliciting an immune response in the egg-producing animal by primary immunization.
3. Administering booster vaccines of one or more antigens of appropriate dosage to induce and maintain the hyperimmune state.

Step 1: The critical point in this step is that the antigen(s) must be capable of inducing immune and hyperimmune states in the egg-producing animal.

Step 2: The vaccine can be administered by any method that elicits an immune response. It is preferred that immunization be accomplished by administering the vaccine through intramuscular or subcutaneous injection. The preferred muscle for injection in an avian is the breast muscle. Dosage is preferably 0.05-5 milligrams of the immunogenic vaccine. Other methods of administration that can be used include intravenous injection, intraperitoneal injection, intradermal, rectal suppository, aerosol or oral administration.

It can be determined whether the vaccine has elicited an immune response in the egg-producing animal through a number of methods known to those having skill in the art of immunology. Examples of these include enzyme-linked immunosorbent assays (ELISA), tests for the presence of antibodies to the stimulating antigens, and tests designed to evaluate the ability of immune cells from the host to respond to the antigen. The minimum dosage of antigen necessary to induce an immune response depends on the vaccination procedure used, including the type of adjuvants and formulation of antigen(s) used as well as the type of egg-producing animal used as the host.

Sten 3: The hyperimmune state is preferably induced and maintained in the target animal by repeated booster administrations of an appropriate dosage at fixed time intervals. The time intervals are preferably one week to three month intervals over a period of 6-12 months. However, it is essential that the booster administrations do not lead to immune tolerance. Such processes are well known in the art. Methods of preparing the hyperimmunized egg product are described, for example, in U.S. Pat. No. 6,803,035 (Greenblatt et al., 2004), which is incorporated by reference herein in its entirety.

In a particular embodiment, an antigen as described herein is formulated into a vaccine containing an adjuvant. The adjuvant can selected from among Freund's complete adjuvant, Freund's incomplete adjuvant, a saponin, a biodegradable polymer, aluminum hydroxide, mineral oil, a surfactant, and combinations thereof. Exemplary saponins include QS-21 and Quil A. Exemplary biodegradable polymers include chitosan, zymosan, a poly(ethylene glycol)-block-poly(propylene glycol)-block-poly(ethylene glycol), such as Pluronic® L121 block copolymer, poly(lactic acid), poly(glycolic acid), poly(lactic-co-glycolic acid), polycaprolactone, and combinations thereof. Exemplary surfactants include polysorbate 80 and sorbitan trioleate. In some embodiments, an antigen as described herein is formulated with an adjuvant selected from the group consisting of Freund's complete adjuvant, Freund's incomplete adjuvant and QS-21 saponin. In the first vaccination, the egg-producing animal receives two 0.5 mL doses of each antigen. Two weeks later, one 0.5 mL dose of each antigen is administered to the egg-producing animal as a booster vaccination. An additional booster vaccination is performed 4 weeks after the first vaccination. The vaccines can be administered intramuscularly. The vaccines can be administered to breast tissue.

It is possible to use other hyperimmunization maintenance procedures or combination of procedures, such as, for example, intramuscular injection for primary immunization and intravenous injection for booster injections. Further procedures include simultaneously administering microencapsulated and liquid antigen, or intramuscular injection for primary immunization, and booster dosages by oral administration or parenteral administration by microencapsulation means. Several combinations of primary and hyperimmunization are known to those skilled in the art.

The hyperimmunized egg or hyperimmunized egg product can contain an increased level of an antibody (e.g. an IgY antibody) specific to a particular antigen disclosed herein relative to a control egg or control egg product obtained from an egg-producing animal that is not hyperimmunized with the particular antigen. In a particular embodiment, the antibody is an IgY antibody.

In some embodiments, the hyperimmunized egg or egg product comprises at least 10%, 20%, 30%, 40%, 50%, 100%, 200%, 300%, 400% or 500% more antibody (e.g. IgY antibody) specific to a particular antigen disclosed herein by weight relative to a control egg or control egg product obtained from an egg-producing animal that is not hyperimmunized with the particular antigen.

The hyperimmunized egg or hyperimmunized egg product can contain increased levels of antibodies to two or more of the antigens disclosed herein, relative to a control egg or control egg product obtained from an egg-producing animal that is not hyperimmunized.

Comparisons of antibody titers in hyperimmunized egg products and control egg products can be determined by methods known in the art. For example, in one embodiment, eggs are collected and the antibody titers are monitored by ELISA at regular intervals. To determine antibody titers, total IgY is extracted from eggs using Pierce™ Chicken IgY Purification Kit (Thermo Fisher Scientific, Waltham, MA). Briefly, 2 mL of egg is mixed with five volumes of delipidation reagent and IgY is purified following the manufacturer's instructions. Spray dried egg powder samples are reconstituted in sterile PBS at 1 mg/mL, and filtered through a 0.22 μm membrane filter. Specific antibody titers in the isolated IgY or egg powder samples are measured by ELISA. Flat bottom, 96-well microtiter plates (Corning® Costar®, Corning, NY) are coated with purified recombinant proteins (e.g. Antigens B, C, Co1, or Co2) at 10 μg/mL (100 μL/well) and incubated overnight at 4° C. The plates are washed twice with PBS containing 0.05% Tween 20 (Sigma-Aldrich, St. Louis, MO) and blocked with 100 μL/well of PBS containing 1% Bovine Serum Albumin (BSA) and incubated for 1 h at RT. Serially diluted (in PBS with 0.1% BSA) IgY samples from egg powder samples are added to the plates in triplicate wells (100 μL/well) and incubated for 2 h at RT with constant shaking. The plates are then washed with PBS-T and treated with peroxidase-conjugated rabbit anti-chicken IgY (IgG) antibody (1:500; Sigma), incubated for 30 min, followed by color development for 10 minutes with 0.01% tetramethylbenzidine substrate (Sigma) in 0.05 M Phosphate-Citrate buffer, pH 5.0. Bound antibodies are detected by measuring optical density at 450 nm (OD₄₅₀) using a microplate reader (Bio-Rad, Hercules, CA).

Antibody titers can be expressed by the highest fold dilution of egg product that still contains detectable antibodies as measured by optical density as described above. For example, an antibody titer of 1000 would indicate that a 1000-fold dilution of the egg product contains detectable antibody, but higher dilutions do not contain detectable antibody. In some embodiments, the antibody titer in the hyperimmunized egg product is at least 100,000, at least 250,000, at least 500,000, or at least 1 million, 2 million, 3 million, 4 million, 5 million, 6 million, 7 million, 8 million, 9 million, 10 million, 11 million, 12 million, 13 million, 14 million, 15 million, 16 million, 17 million, 18 million, 19 million, or 20 million.

In some embodiments, the hyperimmunized egg or egg product comprises at least 0.0001%, 0.0005%, 0.001%, 0.005%, 0.01%, 0.05%, or 0.1% by weight of an IgY antibody to a specific antigen. Typically, a whole chicken egg weighs approximately 60 grams without the shell, with the egg yolk weighing approximately 20 grams and the egg white weighing approximately 40 grams. In some embodiments, 3 grams of egg yolk contains approximately 25 milligrams of total IgY, such that a whole egg contains about 150-200 mg total IgY. In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 25% or 30% of the total IgY in the hyperimmunized egg or egg product is specific to one of the antigens used for hyperimmunization.

Hyperimmunized eggs or egg products can contain an increased level of two or more antibodies (e.g. IgY antibodies), each of which is specific to a different antigen disclosed herein, relative to a control egg or egg product obtained from an egg-producing animal that is not hyperimmunized. The level of increase of each antibody (e.g. IgY antibody) in the hyperimmunized egg or egg product can be at least 10%, 20%, 30%, 40%, 50%, 100%, 200%, 300%, 400%, 500% or more by weight, relative to a control egg or egg product.

### Compositions and Administration

Once the egg-producing animals have been sufficiently hyperimmunized, the eggs from these animals can be collected and processed to produce a hyperimmunized egg product in administrable form. The hyperimmunized egg product can be prepared by dehydration, spray drying, or freeze drying of whole egg, yolk or a purified IgY fraction. The dried hyperimmunized egg product can be mixed with an agent such as silicon or silicon derivatives that improves flow properties. The dried hyperimmunized egg product can comprise a desiccant, or can be stored in a container that includes a desiccant. The hyperimmunized egg product can be stored at ambient temperature or can be refrigerated, for example, at 4° C.

In some embodiments the hyperimmunized egg product can be encapsulated. In some embodiments the nanoparticles provided herein can be encapsulated. Methods of encapsulating antibodies and other proteins are known in the art and are described, for example, in U.S. Pat. No. 7,105,158 (D'Souza et al., 2006). Materials that are biodegradable and nonantigenic can be used as the encapsulating material. Encapsulating materials include, but are not limited to albumin, PLGA, globulin, natural and synthetic polymers, and thermoplastic polymers. Any polymer that is biocompatible and bioerodible can be used for encapsulation. A number of available crosslinking agents such as glutaraldehyde can be used to crosslink the encapsulating material. Additionally, the nanospheres provided herein can be contained with microspheres, where the microspheres are provided as two or more populations, with each population of microspheres having different level of crosslinking, thereby creating a prolonged continuous release of the nanospheres containing the polypeptide, such as IgY.

### Microparticle and Nanoparticle Formulations

In certain aspects, the present disclosure relates to a nanoparticle comprising a composition comprising a polypeptide, wherein the composition is encapsulated in a biocompatible bioerodible polymer and surface-active polymer, such as PVA, polyvinylpyrrolidone, or combinations thereof. Nanoparticles are colloidal particles that are nanometers in size range. Due to their smaller size, the surface area is greater which tends to provide higher loading efficiency and further enhances the bioavailability of the polypeptide contained within the nanoparticles.

In some embodiments, the polypeptide is a therapeutic polypeptide, i.e. a polypeptide that is administered to a subject for the treatment of a disorder. In some embodiments, the polypeptide is an antibody, e.g. a polyclonal antibody or a monoclonal antibody. In some embodiments, the antibody is IgY. In some embodiments, the IgY is obtained from a hyperimmunized egg. In some embodiments, the composition that is encapsulated in the nanoparticle is a hyperimmunized egg product containing IgY. In some embodiments, the composition contained within the nanoparticle is a defatted fraction from egg yolk comprising at least 95 wt %, 96 wt %, 97 wt %, 98 wt %, or 99 wt % IgY based on the total weight of the defatted egg yolk fraction. In some embodiments, the composition contained within the nanoparticle includes a purified IgY.

The nanoparticles can be prepared using one or more polymers. Suitable polymers include, but are not limited to, polyacrylates, polymethacrylates, polycarbonates, polypropylenes, polyalkylenes, polyalkylene glycols, polyalkylene oxides, polyalkylene terephthalates, polyvinyl ethers, polyvinyl halides, polysiloxanes, polyurethanes and copolymers thereof, hydroxyalkyl celluloses, cellulose ethers, nitro celluloses, methyl cellulose, ethyl cellulose, cellulose acetate, cellulose propionate, cellulose acetate butyrate, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenylmethacrylate), poly(methyl acrylate), poly(isopropyl acrylate), poly(isobutylacrylate), poly(octadecyl acrylate), polyethylene, poly(ethylene terephthalatepoly(vinyl acetate), and poly vinyl chloride polystyrene, and mixtures, copolymers, and blends thereof. In a particular embodiment, the polymer includes a polymethacrylate.

In some embodiments, the polymer used to form the nanoparticle is EUDRAGIT® L 100 (methacrylic acid copolymer L (methacrylic acid-methyl methacrylate copolymer (1:1), or methacrylic acid copolymer, Type A, available from Evonik Corporation, Allentown, PA). The ratio of the free carboxyl groups to the ester groups is about 1:1 in EUDRAGIT® L 100.

In some embodiments, the polymer used to form the nanoparticle or microparticle is EUDRAGIT® S 100 (methacrylic acid copolymer S (methacrylic acid-methyl methacrylate copolymer (1:2), or methacrylic acid copolymer, Type B, available from Evonik Corporation, Allentown, PA). The ratio of the free carboxyl groups to the ester groups is about 1:2 in EUDRAGIT® S 100.

In some embodiments, a combination of EUDRAGIT® L 100 and EUDRAGIT® S 100 can be used to form the nanoparticle or microparticle. The ratio of EUDRAGIT® L 100 to EUDRAGIT® S 100 can be in the range of 99:1 to 1:99. In some embodiments, the ratio of EUDRAGIT® L 100 to EUDRAGIT® S 100 can be 80:20 to 20:80, or 75:25 to 25:75, or 60:40 to 40:60. In some embodiments, EUDRAGIT® L 100 is used in excess of EUDRAGIT® S 100 (there is a greater wt % of EUDRAGIT® L 100 than EUDRAGIT® S 100). In some embodiments, the ratio of EUDRAGIT® L 100 to EUDRAGIT® S 100 is in a range of 2:1 to 10:1. In some embodiments, EUDRAGIT® S 100 is used in excess of EUDRAGIT® L 100 (there is a greater wt % of EUDRAGIT® S 100 than EUDRAGIT® L 100). In some embodiments, the ratio of EUDRAGIT® S 100 to EUDRAGIT® L 100 is in a range of 2:1 to 10:1. In some embodiments, equal amounts of EUDRAGIT® L 100 and EUDRAGIT® S 100 are used.

A pH-responsive release profile for the nanoparticles can be achieved by the appropriate selection of one or a combination of polymers as the biocompatible bioerodible polymer used to form the nanoparticles.

A surface-active polymer also can be included when forming the nanoparticle or microparticle. Exemplary surface-active polymers include poly(vinyl) alcohol PVA, polyvinylpyrrolidone (PVP), and combinations thereof. In some embodiments, the surface-active polymer includes PVA.

Encapsulating IgYs in nanoparticles makes them more stable, and they can remain in their native structure without being degraded. With use of nanoparticles, the IgY entrapped inside the nanoparticle is safe in gastric fluid as well as intestinal fluids, and is protected from enzymatic degradation. Being entrapped inside the nanoparticle, the molecular weight and large size of IgYs is no longer a problem since the overall size of the nanoparticle is in the nano range and therefore, it can more easily be presented to and pass through the cells or interstitial regions and reach the target site.

The nanoparticles can have any shape. Typically the nanoparticles are spherical. Other suitable shapes include, but are not limited to, flakes, triangles, ovals, rods, polygons, needles, tubes, cubes and cuboid structures. In certain embodiments, the nanoparticles have a diameter of less than 1000, 900, 800, 700, 600, 500, 400, 300, 200, 100, 50, 25, or 10 nm. Any of these values can be used to define a range for the diameter of the nanoparticle. For example, the diameter of the nanoparticle can be from about 10 to about 1000 nm, from about 100 to about 1000 nm, or from about 50 to about 500 nm, or from about 10 to about 100 nm, from about 200 to about 300 nm, from about 250 to about 300 nm, or from about 100 to about 400 nm. In certain embodiments, the nanoparticles have a diameter of less than 300 nm.

The concentration of polypeptide (e.g. IgY) encapsulated in a nanoparticle can be presented as percent loading, i.e. the ratio of the weight of polypeptide (e.g. IgY) to the weight of the nanoparticle, multiplied by 100. In some embodiments, the nanoparticle comprises about 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9, 10, 11, 12, 13, 14, 15, 20, 25, 30, 35, 40, 45 or 50 wt % polypeptide (e.g. IgY) with respect to the total weight of the nanoparticle. In some embodiments, the nanoparticles comprise less than about 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9, 10, 11, 12, 13, 14, 15, 20, 25, 30, 35, 40, 45 or 50 wt % polypeptide (e.g. IgY) with respect to the total weight of the nanoparticle. In some embodiments, the nanoparticles comprise at least about 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9, 10, 11, 12, 13, 14, 15, 20, 25, 30, 35, 40, 45 or 50 wt % weight polypeptide (e.g. IgY) with respect to the total weight of the nanoparticle. Any of these values can be used to define a range for the concentration of the polypeptide (e.g. IgY) in the nanoparticle. For example, the nanoparticles can contain the polypeptide (e.g. IgY) in a concentration ranging from about 1 wt % to about 10 wt %, or from about 1 wt % to about 5 wt %. In some embodiments, the concentration of polypeptide (e.g. IgY) in the nanoparticles is at least about 5 wt %.

In some embodiments, the nanoparticle comprising the polypeptide (e.g., IgY) is anionic, e.g., has a negative zeta potential. In some embodiments the zeta potential of the nanoparticles is less than 0, −1, −5, −10, −15, −20, −25, −30, −31, −32, −33, −34, −35, −36, −37, −38, −39, −40, −45 or −50 mV. Any of these values may be used to define a range for the zeta potential of the nanoparticle. For example, in some embodiments, the zeta potential of the nanoparticle is from about 0 to about −40 mV, about 0 to about −50 mV, about −10 to about −50 mV, about −20 to about −50 mV, or about −30 to about −40 mV. In some embodiments, the zeta potential of the nanoparticle is about −38.9 mV. In some embodiments, the zeta potential of the nanoparticle is about −31.2 mV.

There are several processes whereby nanoparticles can be prepared, including, for example, multi-walled microencapsulation, hot melt encapsulation, phase separation encapsulation, spontaneous emulsion, solvent evaporation microencapsulation, solvent removal microencapsulation, and coacervation. These methods are known in the art. Detailed descriptions of the methods are discussed in Mathiowitz et al., “Microencapsulation”, in Encyclopedia of Controlled Drug Delivery, vol. 2, pp. 495-546, 1999, John Wiley & Sons, Inc. New York, N.Y., which is incorporated by reference herein in its entirety.

Provided is a method for preparing nanoparticles that include a polypeptide having a molecular weight greater than 50,000 g/mol. For example, in some embodiments, the molecular weight of the polypeptide (e.g., an IgY) encapsulated in the nanoparticle is at least 50,000, 60,000, 70,000, 80,000, 90,000, 100,000, 110,000, 120,000, 130,000, 140,000, 150,000, 160,000, 170,000, 180,000, 190,000 or 200,000 g/mol. In some embodiments, the molecular weight of the polypeptide (e.g., an IgY) encapsulated in the nanoparticle is from 50,000 to 100,000 g/mol, from 50,000 to 180,000 g/mol, or from 50,000 to 200,000 g/mol. In some embodiments, the molecular weight of the polypeptide (e.g., an IgY) encapsulated in the nanoparticle is about 180,000 g/mol.

A combination of homogenization processes is used for preparing the nanoparticles. A probe homogenizer can be used to make a first emulsion, and the first emulsion then can be processed in a high-pressure homogenizer.

A probe homogenizer is a rotor stator homogenizer that includes a rotating blade member (rotor) and a stationary collar that contains perforations (stator). The rotating blade forces material through the perforations and the shear forces between the perforations in the stator and the rotating blade as it passes the perforations reduces particle size. Because of the motion of the blade, the material is drawn into the rotor/stator assembly, which also results in cavitation, which also can help to reduce particle size. The size of the perforations in the stator, the rotational speed of the rotating blade member, the viscosity of the material, and the length of time of processing can influence the resulting particle size. Probe homogenizers of various dimensions and configurations of rotors and stators are known in the art and are commercially available, and can be used in the methods provided herein.

The emulsion produced using the probe homogenizer then can be processed in a high-pressure homogenizer (HPH). The high-pressure homogenizer converts fluid pressure into kinetic energy. Typical high-pressure homogenizers include a homogenization valve through which a material is forced to flow, and a high-pressure pump to force the material through the valve. The high pressure homogenization process exposes the material being processed to multiple forces such as shear, cavitation, turbulence, impact forces, and high pressure, which aid in formation of the nanoparticles, and can result in nanoparticles with optimal characteristics like minimal particle size, good particle size distribution, and maximum polypeptide loading. High pressure homogenizers of various dimensions and configurations are known in the art and are commercially available, and can be used in the methods provided herein.

In an exemplary embodiment, an aqueous phase can be prepared to contain a composition that includes the polypeptide (e.g., IgY), and a surface-active polymer. The surface-active polymer can be any one of poly(vinyl) alcohol (PVA), polyvinylpyrrolidone (PVP), or a combination thereof. An organic phase then is prepared. The organic phase can include absolute ethanol, dichloromethane (DCM), acetonitrile, acetone, ethyl acetate, chloroform, and combinations thereof. In some methods, the organic phase includes DCM. A solution or suspension containing a biocompatible bioerodible polymer is prepared. To assist in mixing, the aqueous phase or solution or suspension of biocompatible bioerodible polymer or both can be subjected to sonication. The aqueous phase, organic phase, and solution or suspension of biocompatible bioerodible polymer then can be mixed together and subjected to emulsification using a probe homogenizer (rotor/stator homogenizer). The configuration and time of mixing can depend on the volume of material being processed. In some methods, the mixture is subjected to homogenization using the probe homogenizer at a rotor rotational speed of 5,000 to 15,000 rpm. The time for processing can be from 1 to 10 minutes. A cooling element can be included during probe homogenization. For example, the vessel containing the materials being processed can be placed in an ice bath, or a refrigeration system can be used to remove heat from the material being processed during probe homogenization. The treatment with the probe homogenizer results in a coarse emulsion.

The coarse emulsion then is subjected to a further homogenization process using a high-pressure homogenizer. The pressure used can be from about 5,000 psi to 45,000 psi. In some embodiments, the method includes processing in a high-pressure homogenizer at a pressure of about 15,000 psi.

The resulting emulsion from the high-pressure homogenizer then can be subjected to solvent removal. In some embodiments, a rotary evaporator can be used to remove the solvents. Any rotary evaporator known in the art can be used to remove the solvents. In some embodiments, a rotary vacuum evaporator can be used. The time for evaporation of the solvent can depend on the amount of solvent present and the chemical properties of the solvent. In some embodiments, only the organic solvent is removed, leaving an aqueous suspension of the nanoparticles. In some methods, rotary vacuum evaporation at a pressure of from about 50 to 250 psi is used. In some methods, rotary vacuum evaporation at a pressure of about 200 psi for about 10 minutes is used to remove the organic solvents. In some methods, the emulsion from the high-pressure homogenizer can turn transparent after rotary vacuum evaporation, indicating that the organic solvents have been removed.

The material obtained after removal of the organic solvents can be subjected to centrifugation to recover the nanoparticles. In some methods, a two-step centrifugation process can be used. In a typical two-step centrifugation process, the obtained material can first be centrifuged at 3,000-5,000×g for a period of from 2 to 10 minutes. In some embodiments, the material obtained after solvent removal can be centrifuged at 4,400×g for about 3 min. After the first centrifugation step, the supernatant can collected and centrifuged at 20,000-25,000×g for a period of 10 to 30 minutes using a high-speed centrifuge, such as an Avanti® J-E high-speed centrifuge. In some methods, after the first centrifugation step, the supernatant can collected and centrifuged at about 22,000×g for a about 15 minutes using a high-speed centrifuge. The pelleted nanoparticles can be resuspended in Milli-Q® IQ water ((produced using a Milli-Q® IQ water purification system, available from MilliporeSigma, St. Louis, MO)).

In some embodiments, the polypeptide (e.g. IgY) in a concentrated form is encapsulated in the nanoparticle. For example, in some embodiments, the polypeptide (e.g. IgY) is purified or partially purified and concentrated before formation of the nanoparticles. Methods of purifying and concentrating IgY antibodies from egg products are known in the art and are described, for example, in U.S. Pat. No. 5,367,054 (Lee, 1994), which is incorporated by reference herein in its entirety.

In certain embodiments, the nanoparticles compositions containing the nanoparticles disclosed herein can be administered to the subject by oral administration. Egg and egg yolk are natural food ingredients and are non-toxic and safe for oral consumption. The dosage form containing the nanoparticles or containing a composition containing the nanoparticles provided herein can further comprise a pharmaceutically acceptable carrier suitable for oral administration. In some embodiments, the pharmaceutically acceptable carrier comprises a compound that is generally recognized as safe (GRAS) by the FDA. In some embodiments, the GRAS compound is selected from acetic acid, aconitic acid, adipic acid, alginic acid, α-amylase enzyme preparation from Bacillus stearothermophilus, benzoic acid, bromelain, caprylic acid, mixed carbohydrase and protease enzyme product, citric acid, catalase (bovine liver), lactic acid, enzyme-modified lecithin, linoleic acid, malic acid, potassium acid tartrate, propionic acid, stearic acid, succinic acid, sulfuric acid, tannic acid, tartaric acid, diacetyl tartaric acid esters of mono- and diglycerides, agar-agar, brown algae, red algae, ammonium alginate, ammonium bicarbonate, ammonium carbonate, ammonium chloride, ammonium hydroxide, ammonium citrate, dibasic, ammonium phosphate (monobasic), ammonium phosphate (dibasic), ammonium sulfate, bacterially-derived carbohydrase enzyme preparation, bacterially-derived protease enzyme preparation, bentonite, benzoyl peroxide, n-butane and iso-butane, calcium acetate, calcium alginate, calcium carbonate, calcium chloride, calcium citrate, calcium gluconate, calcium glycerophosphate, calcium hydroxide, calcium iodate, calcium lactate, calcium oxide, calcium pantothenate, calcium propionate, calcium stearate, calcium sulfate, carbon dioxide, beta-carotene, cellulase enzyme preparation derived from Trichoderma longibrachiatum, clove and its derivatives, cocoa butter substitute, copper gluconate, copper sulfate, corn silk and corn silk extract, cuprous iodide, L-cysteine, L-cysteine monohydrochloride, dextrin, diacetyl, dill and its derivatives, enzyme-modified fat, ethyl alcohol, ethyl formate, ferric ammonium citrate, ferric chloride, ferric citrate, ferric phosphate, ferric pyrophosphate, ferric sulfate, ferrous ascorbate, ferrous carbonate, ferrous citrate, ferrous fumarate, ferrous gluconate, ferrous lactate, ferrous sulfate, ficin, garlic and its derivatives, glucono delta-lactone, corn gluten, wheat gluten, glyceryl monooleate, glyceryl monostearate, glyceryl behenate, glyceryl palmitostearate, acacia (gum arabic), gum ghatti, guar gum, locust (carob) bean gum, karaya gum (sterculia gum), gum tragacanth, gellan gum, xanthan gum, hydrogen peroxide, inositol, insoluble glucose isomerase enzyme preparations, iron, elemental, isopropyl citrate, lactase enzyme preparation from Candida pseudotropicalis, lactase enzyme preparation from Kluyveromyces lactis, lecithin, licorice and licorice derivatives, ground limestone, animal lipase, lipase enzyme preparation derived from Rhizopus niveus, magnesium carbonate, magnesium chloride, magnesium hydroxide, magnesium oxide, magnesium phosphate, magnesium stearate, magnesium sulfate, malt, maltodextrin, malt syrup (malt extract), manganese chloride, manganese citrate, manganese gluconate, manganese sulfate, menhaden oil, methylparaben, microparticulated protein product, mono- and diglycerides, monosodium phosphate derivatives of mono- and diglycerides, niacin, niacinamide, nickel, nisin preparation, nitrogen, nitrous oxide, peptones, rapeseed oil, ox bile extract, ozone, pancreatin, papain, pectins, pepsin, potassium alginate, potassium bicarbonate, potassium carbonate, potassium chloride, potassium citrate, potassium hydroxide, potassium iodide, potassium iodate, potassium lactate, potassium sulfate, propane, propyl gallate, propylene glycol, propylparaben, pyridoxine hydrochloride, rennet (animal-derived) and chymosin preparation (fermentation-derived), riboflavin, riboflavin-5-phosphate (sodium), rue, oil of rue, shea nut oil, sodium acetate, sodium alginate, sodium benzoate, sodium bicarbonate, sodium carbonate, sodium citrate, sodium diacetate, sodium hydroxide, sodium hypophosphite, sodium lactate, sodium metasilicate, sodium propionate, sodium sesquicarbonate, sodium tartrate, sodium potassium tartrate, sodium thiosulfate, sorbitol, stannous chloride (anhydrous and dihydrated), starter distillate, stearyl citrate, sucrose, corn sugar, invert sugar, corn syrup, high fructose corn syrup, thiamine hydrochloride, thiamine mononitrate, α-tocopherols, triacetin, tributyrin, triethyl citrate, trypsin, urea, urease enzyme preparation from Lactobacillusfermentum, vitamin A, vitamin B12, vitamin D, beeswax (yellow and white), candelilla wax, carnauba wax, whey, reduced lactose whey, reduced minerals whey, whey protein concentrate, baker's yeast extract, brewer's yeast extract, zein, and aminopeptidase enzyme preparation derived from Lactococcus lactis, or any combination thereof.

In certain embodiments, the dosage form containing the nanoparticles or a composition containing the nanoparticles provided herein can include one or more additional compounds, e.g. a nutrient or probiotic. For example, in one embodiment, the nanoparticles or a composition containing the nanoparticles can be integrated into a dietary supplement.

One method for preparing the hyperimmunized egg to be incorporated into nanoparticle involves drying the egg into an egg powder. Various methods are known for drying eggs, including freeze-drying and spray drying. In one method, spray drying is a used. The process of spray drying eggs is well known in the art.

In certain embodiments, whole eggs are divided into separate fractions such as egg yolks and egg whites. For example, it is generally known in the art that IgY antibody is found in egg yolks. Accordingly, those having ordinary skill in the art would clearly recognize that separation of egg yolks could provide more potent fractions or elimination of undesirable components. Such further separation will provide for the ability to make nanoparticles that include the more potent egg fraction. In some embodiments, the IgY is separated from a hyperimmunized egg product and included in the nanoparticles. The separated IgY can be processed into a freeze-dried powder or spray-dried powder to be included in nanoparticles. The IgY can be further purified prior to being incorporated into nanoparticles.

In certain embodiments, the disclosure relates to a method of delivering polypeptide (e.g. IgY) to a subject, comprising administering a nanoparticle or a composition containing a nanoparticle as described herein to the subject by oral administration. The nanoparticle or composition containing the nanoparticle is preferably administered to the subject in an amount that is effective in treating or preventing a particular disorder. Dosage and duration of the administration will depend upon the particular condition of the subject. In some embodiments, the nanoparticle or composition containing the nanoparticle is administered to the subject for at least 5, 6, 7, 8, 9, 10, 11, 12, 13, 14, 15, 20, 25, 30, 60, 90, 180 or 365 days. The nanoparticle or composition containing the nanoparticle can be administered to the subject 1, 2, 3, 4, 5, 6, 7, 8, 9, 10 or more times per day. Any of these values can be used to define a range for the number of times the composition can be administered to the subject per day. For example, in some embodiments the nanoparticle or composition containing the nanoparticle is administered to the subject 1-2 times per day, 1-3 times per day, or 1-4 times per day. In some embodiments, the nanoparticle or composition containing the nanoparticle is administered to the subject at least twice per day. In some embodiments, the nanoparticle or composition containing the nanoparticle is administered to subject at least once per day.

In some embodiments, the composition is administered to the subject daily. In some embodiments, the nanoparticle or composition containing the nanoparticle is administered to the subject once every two days. In some embodiments, the nanoparticle or composition containing the nanoparticle is administered to the subject once every three days. In some embodiments, the nanoparticle or composition containing the nanoparticle is administered to the subject once per week. In a particular embodiment, the nanoparticle or composition containing the nanoparticle is administered to the subject once per day for more than 10 consecutive days. Also provided are uses of the nanoparticles or compositions containing the nanoparticle as described herein for delivering a polypeptide to a subject.

In some embodiments, daily amounts having an equivalent ranging from less than one to several whole, hyperimmune eggs (or hyperimmune egg products containing the equivalent of less than one to several whole, hyperimmune eggs) can be administered to the subject depending on the particular circumstance of the condition.

In certain embodiments, the effective amount of the hyperimmunized egg product in the form of nanoparticles administered to a subject (e.g. a human) is 0.1, 0.2, 0.3, 0.4, 0.5, 0.6, 0.7, 0.8, 0.9, 1, 2, 3, 4, 5, 6, 7, 8, 9, 10, 20, 30, 40 or 50 grams per day. For example, in some embodiments, 0.1, 0.2, 0.3, 0.4, 0.5, 0.6, 0.7, 0.8, 0.9, 1, 2, 3, 4, 5, 6, 7, 8, 9, 10, 20, 30, 40 or 50 grams per day of whole egg nanoparticles are administered to the subject. In some embodiments, 0.1, 0.2, 0.3, 0.4, 0.5, 0.6, 0.7, 0.8, 0.9, 1, 2, 3, 4, 5, 6, 7, 8, 9, 10, 20, 30, 40 or 50 grams per day of egg yolk nanoparticles are administered to the subject. In some embodiments, 0.1, 0.2, 0.3, 0.4, 0.5, 0.6, 0.7, 0.8, 0.9, 1, 2, 3, 4, 5, 6, 7, 8, 9 or 10 grams per day of dried egg yolk or dried whole egg nanoparticles are administered to the subject. Any of these values can be used to define a range for the effective amount of the nanoparticles containing hyperimmunized egg product administered to the mammal. For example, in some embodiments the effective amount of the nanoparticles containing hyperimmunized egg product is between 0.1 and 10 grams, between 0.5 to 6 grams, or between 1 and 5 grams per day. In a particular embodiment, an amount of nanoparticle containing IgY equivalent to the amount of IgY in 3 grams of egg yolk is administered to the subject (e.g. a human) per day.

### EXAMPLES

### Example 1. IgY Quantification Using NanoDrop™ One

IgY and serial dilutions of IgY ranging from 0.105-26.9 mg/mL) were analyzed for A280 (absorption at 280 nm) values using a NanoDrop™ One Microvolume UV-Vis Spectrophotometer (Thermo Scientific). Sample type was selected as IgY and baseline correction was performed at 320 nm. 2 μL of 1× phosphate buffered saline (PBS) was placed onto the lower pedestal and the lower arm for blanking (Auto-blank was on). Samples were gently vortexed before taking measurements. 2 μL aliquots of each of the samples were loaded and respective A280 values were recorded. All the sample dilutions were carried out using 1×PBS. The A280 values are shown in in Table 1.

TABLE 1
IgY Quantification Results

| | A280 | | |
|---|---|---|---|
| Theoretical Amount | (absorption at 280 nm) | | |
| (mg/mL) | T1 | T2 | Average |
| 26.9 | 37.886 | 37.895 | 37.8905 |
| 13.45 | 19.981 | 18.403 | 19.192 |
| 6.725 | 9.836 | 8.954 | 9.395 |
| 3.3625 | 4.804 | 4.546 | 4.675 |
| 1.68125 | 2.27 | 2.162 | 2.216 |
| 0.840625 | 1.041 | 1.074 | 1.0575 |
| 0.420313 | 0.5 | 0.461 | 0.4805 |
| 0.210156 | 0.214 | 0.236 | 0.225 |
| 0.105078 | 0.07 | 0.077 | 0.0735 |

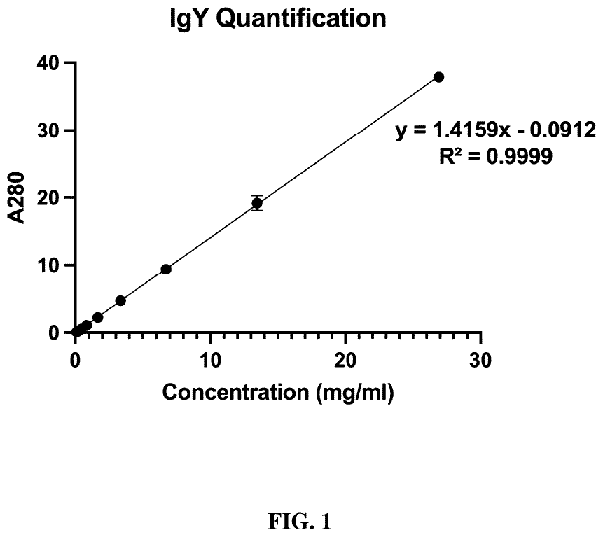

The results are shown graphically in FIG. 1, which provides a calibration curve for IgY quantification. The R² value was determined to be 0.9999.

### Example 2. Preparation of IgY Nanoparticles

Nanoparticles containing IgY antibodies were prepared. To achieve efficient loading of IgY into nanoparticles, a high-pressure homogenizer (HPH) was utilized. The high-pressure homogenization process facilitates the formulations' exposure to multiple forces such as shear, cavitation, turbulence, impact forces, and high pressure which aid in accomplishing nanoparticles with optimal characteristics like minimal particle size and maximum drug loading. EUDRAGIT® L 100, either alone or in combination with EUDRAGIT® S 100 was used for the preparation of IgY nanoparticles. An aqueous phase containing IgY solution equivalent to 20 mg of IgY and 1% polyvinyl alcohol (PVA) was prepared. The PVA was used as the surface-active polymer. An organic phase containing 3 mL of dichloromethane (DCM) and 1 mL of absolute ethanol was prepared. The EUDRAGIT® polymer(s) was/were dissolved in absolute ethanol using bath sonication. The solution containing IgY and PVA, the solution containing DCM, and the alcoholic solution of the EUDRAGIT® polymer(s) were subjected to probe homogenization using a digital high-speed homogenizer at 10,000 rpm for 1 minute (Fisherbrand™ 850 Homogenizer, Fisher Scientific, Waltham, MA). The resulting coarse emulsion was processed through the HPH (NanoDeBee homogenizer, BEE International, San Diego, CA)). The detailed HPH parameters used for homogenization were as follows: nozzle type: N5; pressure: 15,000 psi; flow pattern: reverse; number of reactors: 6; number of cycles: 7. During high pressure homogenization, Milli-Q® water (produced using a Milli-Q® IQ water purification system, available from MilliporeSigma, St. Louis, MO) was added to collect 45 mL of preparation. Then, formulations were subjected to rotary vacuum evaporation at about 200 psi for about 10 minutes to remove the organic solvents. Endpoint was determined when the solution turned transparent. The obtained solutions were centrifuged at 4,400×g for 3 min. The supernatant was collected and centrifuged for 15 minutes at 22,000×g using an Avanti® J-E high-speed centrifuge (Beckman Coulter Life Sciences, Indianapolis, IN). The collected nanoparticles were resuspended in 1 mL of Milli-Q® water.

### Example 3. Physicochemical Characterization of IgY Nanoparticles

The IgY nanoparticles were analyzed for their physicochemical characteristics. The IgY nanoparticles were frozen at −80° C. overnight. Then, frozen IgY nanoparticles were subjected to freeze-drying using L Benchtop Freeze Dry system for 24 hours. Lyophilized IgY nanoparticle powder was collected and analyzed for the IgY amount by dissolving known amount on pH 7.4 buffer followed by NanoDrop™ One analysis.

10 μL of nanoparticle formulation was diluted with 2 mL of Milli-Q® water and sonicated for 30 minutes. Particle size, polydispersity index (PDI) and zeta potential using Malvern Zeta sizer were measured. % entrapment efficiency (% EE) of IgY loaded nanoparticles also was determined.

The IgY nanoparticles formulated with EUDRAGIT® L 100 (IgY-L nanoparticles) were found to have an average particle size of 276.0 nm and polydispersity index (PDI) of 0.22. The IgY nanoparticles formulated with EUDRAGIT® L 100/S 100 (IgY-L:S nanoparticles, where the ratio of L100:S100 was 4:1) were found to have an average particle size of 272.1 nm and polydispersity index (PDI) of 0.17. These data suggest the IgY nanoparticles have a uniform particle size distribution.

The zeta potential of IgY-L nanoparticles was found to be −38.9 mV, and the zeta potential of the IgY-L:S nanoparticles was found to be −31.2 mV. Accordingly, the nanoparticles were anionic. Anionic nanoparticles have greater stability during storage, and greater safety during administration to humans or animals since cationic nanoparticles may cause toxicity and cell death.

IgY was effectively encapsulated into nanoparticles, achieving % encapsulation efficiency (EE) of 35.03±3.8% for the IgY-L nanoparticles and 36.4±1.99% for the IgY-L:S nanoparticles. The encapsulation efficiency is the percentage of IgY in the sample that was encapsulated in the nanoparticles.

### Example 4. ELISA Assay

A functional ELISA assay was performed using the IgY extracted from the nanoparticles and was compared with control IgY at various concentrations. IgY loaded nanoparticle formulations (IgY-L and IgY-L:S nanoparticles) were lysed and the lysis samples were diluted to obtain a concentration of 300 ng/mL for IgY. Then, the diluted samples were quantified for IgY amount using ELISA assay. In brief, antigen was diluted in bicarbonate buffer pH 9.0 to 1 mg/mL. 100 μL of this antigen solution was added to ELISA plate wells in a 96 well plate. Then, plate was covered with parafilm and incubated overnight at 4° C. Then, solution was discarded by tilting/flicking the plate followed by washing it once using 200 μL PBS containing 0.05 wt % Tween-20 (PBST). Then, 200 μL of fresh blocking reagent (1% BSA in 1×PBS) was added in each well and incubated for 1 hour at 37° C. Next, the plate was washed once using PBST for 5 min. Then, it was tilt-tapped on a paper towel. Primary antibody from the stock solution diluted in PBST was added. Serial dilutions of IgY were performed ranging from 37 to 300 ng/mL and the plate were incubated at 37° C. for 1 hour. Then, the plate was washed 3 times with PBST, with each wash for 5 min using 100 μL of PBST without any agitation. Secondary antibody diluted in blocking solution was added and incubated at 37° C. for 1 hour followed by washing twice with PBST and one final wash in PBS only, each wash for 5 min. Then, 100 μL of developing solution (TMB substrate solution) was added and incubated for 15-20 min at room temperature followed by addition of 100 μL of stop solution. The plate was read for the absorbance values using a plate reader at 450 nm.

A calibration on plot was constructed for the quantification of IgY and its serial dilutions using the functional ELISA assay. The equation obtained was y=0.0058x+1.8906. The absorbance values obtained from the lysis samples of IgY nanoparticles were incorporated in the above-mentioned equation to calculate IgY quantities. From the results, it was determined that the IgY-L nanoparticles contained 274.2±6.7 ng/mL of IgY antibody, and the IgY-L:S nanoparticles contained 273.8±6.4 ng/mL of IgY antibody. For the percent loading of the nanoparticles, the IgY-L nanoparticles contained 5.84%±0.64 IgY per weight of the nanoparticle (w/w) and the IgY-L:S nanoparticles contained 6.07%±0.33 IgY per weight of the nanoparticle (w/w).

### Example 5. Preparation of IgY Nanoparticle-Loaded Capsules

Hard gelatin capsules of size #3 were filled with the lyophilized IgY nanoparticles manually. The loaded capsules were coated with either coating formulation A or B. The composition of coating formulation A is shown in Table 2. The composition of coating formulation B is shown in Table 3.

TABLE 2
Composition of Coating Formulation A

| Coating Formulation A | Quantity | |
|---|---|---|
| EUDRAGIT ® L 100 | 0.600 | g |
| Triethyl citrate | 0.060 | g |
| Talc | 0.300 | g |
| Isopropyl alcohol | 5.1 | mL |
| Acetone | 3.4 | mL |
| Milli-Q water | 0.42 | mL |
| Total | 10 | g |

TABLE 3
Composition of Coating Formulation B

| Coating Preparation-A | Quantity | |
|---|---|---|
| EUDRAGIT ® L 100 | 0.048 | g |
| EUDRAGIT ® S 100 | 0.012 | g |
| Triethyl citrate | 0.060 | g |
| Talc | 0.300 | g |
| Isopropyl alcohol | 5.1 | mL |
| Acetone | 3.4 | mL |
| Milli-Q water | 0.42 | mL |
| Total | 10 | g |

The actual quantities of the ingredients for dispensing can be appropriately scaled up depending on the batch size to be manufactured. Required quantities of EUDRAGIT® L100 (0.6 g) [Formulation A] or EUDRAGIT® L100 (0.048 g), EUDRAGIT® S 100 (0.012 g) [Formulation B], talc (0.3 g), triethyl citrate (0.06 g) were weighed. Required amounts of solvents: isopropyl alcohol (5.1 mL), milli-Q water (0.42 mL) and acetone (3.4 mL) were measured. All the weighed ingredients were added to the mixture of solvents mentioned before and subjected for bath sonication for 30 minutes. The resulting coating composition was transferred to a 25 mL glass beaker. Then capsules containing the IgY nanoparticles were dipped into coating formulation. Dip coating was carried out for 3 times with a drying step of 5 minutes between coatings. Then the capsules were dried using an air dryer with hot air flow. The integrity of the coated capsules then was checked and it was determined that the coating process did not impact capsule integrity.

### Example 6. In-Vitro Release Studies

Release studies for coated blank capsules and coated capsules containing IgY (Formulation A: EUDRAGIT® L100, and Formulation B: EUDRAGIT® L 100 and S 100) were conducted in pH 1.2 and pH 7.0 buffers (50 mL volume) and performed at 37° C. The coated capsules loaded with IgY nanoparticles were placed in release media (50 mL) using sinker and 1 mL of samples were collected at pre-determined time points (pH 1.2:1 and 2 hours, pH 7.0:1 hour). After 2 hours at pH 1.2, the buffer was removed and replaced with pH 7.0 buffer. The collected samples were centrifuged for 15 minutes at 30,000 rpm followed by filtration using 0.2 μm syringe filter. Filtrates were analyzed using the NanoDrop™ One spectrophotometer for the determination of the amount of IgY released from the capsules.

The capsule containing the IgY nanoparticles and coated with coating formulation A was found to release about 46.3% of IgY after 1 hour and 99.0% of IgY after 2 hours in pH 1.2 buffer. About 6.5% of IgY was released after 1 hour after replacing the pH 1.2 buffer with pH 7.0 buffer. The capsule containing the IgY nanoparticles and coated with coating formulation B was found to release about 90.7% of IgY after 1 hour and 91.3% IgY after 2 hours in pH 1.2 buffer. About 11.5% of IgY was released after 1 hour after replacing the pH 1.2 buffer with pH 7.0 buffer.

1. A nanoparticle, comprising a composition comprising a polypeptide, wherein the composition is encapsulated in a material comprising a biocompatible bioerodible polymer and a surface-active polymer.

2. The nanoparticle of claim 1, wherein the biocompatible bioerodible polymer is a polymethacrylate.

3. (canceled)

4. The nanoparticle of claim 1, wherein the nanoparticle comprises at least 5% w/w of the polypeptide.

5. The nanoparticle of claim 1, wherein the polypeptide is IgY.

6. The nanoparticle of claim 5, wherein the IgY is obtained from a hyperimmunized egg.

7. The nanoparticle of claim 1, wherein the composition is a hyperimmunized egg product.

8. The nanoparticle of claim 1, wherein the composition is a defatted fraction from egg yolk comprising at least 95% IgY by weight.

9. The nanoparticle of claim 1, wherein the composition comprises a purified IgY.

10. The nanoparticle of claim 1, wherein the biocompatible bioerodible polymer comprises:
(a) methacrylic acid-methyl methacrylate copolymer (1:1); or
(b) methacrylic acid-methyl methacrylate copolymer (1:2); or
(c) both (a) and (b).

11. The nanoparticle of claim 1, wherein the surface-active polymer is selected from among chitosan, poly(vinyl) alcohol (PVA), polyvinylpyrrolidone (PVP), and combinations thereof.

12. A pharmaceutical composition, comprising:
the nanoparticle of claim 1; and
a pharmaceutically acceptable carrier.

13. The pharmaceutical composition of claim 12, wherein the pharmaceutically acceptable carrier is suitable for oral administration.

14. The pharmaceutical composition of claim 12, wherein the pharmaceutical composition is in a form selected from the group consisting of a powder, a tablet, an enterically coated tablet, a capsule, an enterically coated capsule, a suspension, a solution, and an oral beverage.

15. A method of delivering a polypeptide to a subject, comprising: administering the nanoparticle of claim 1 to the subject by oral administration.

16. The method of claim 15, wherein the polypeptide contained in the nanoparticle is IgY.

17-20. (canceled)

21. The method of claim 15, wherein the biocompatible bioerodible polymer of the nanoparticle comprises a polymethacrylate.

22-23. (canceled)

24. A method of preparing the nanoparticle of claim 1, the method comprising:
(a) preparing a first mixture comprising a composition comprising a polypeptide, water, and a surface-active polymer;
(b) sonicating the first mixture;
(c) adding a biocompatible bioerodible polymer to ethanol and sonicating to produce a second mixture;
(d) mixing the first mixture and second mixture with an organic solvent to form a third mixture;
(e) homogenizing the third mixture using a probe homogenizer at a speed of about 5,000 to 15,000 rpm to yield a coarse emulsion;
(f) subjecting the coarse emulsion to high pressure homogenization using a high-pressure homogenizer at a pressure of about 10,000 psi to 30,000 psi to form a fourth mixture; and
(g) evaporating the ethanol and the organic solvent from the fourth mixture to yield a suspension containing the nanoparticle.

25. (canceled)

26. The method of claim 24, wherein the surface-active polymer is selected from among chitosan, poly(vinyl) alcohol (PVA), polyvinylpyrrolidone (PVP), and combinations thereof.

27-28. (canceled)

29. The method of claim 24, wherein the polypeptide is IgY.

30. (canceled)

31. The method of claim 24, wherein:
(a) the surface-active polymer comprises 0.1% (w/w) chitosan; or
(b) the surface-active polymer comprises 0.5 to 2.5% (w/w) poly(vinyl) alcohol (PVA); or
(c) the surface-active polymer comprises 0.5 to 2.5% (w/w) polyvinylpyrrolidone (PVP); or
(d) any combination of (a) through (c).

32-34. (canceled)
